(19) United States
(12) Patent Application Publication
(10) Pub. No.: US 2010/0056906 A1
(43) Pub. Date: Mar. 4, 2010

(54) DISPLACEMENT DETECTION IN OPTICAL TOMOGRAPHY SYSTEMS

(75) Inventor: Willem Peter Van Der Brug, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronics N.V., Eindhoven (NL)

(21) Appl. No.: 12/439,702

(22) PCT Filed: Sep. 3, 2007
(86) PCT No.: PCT/IB2007/053529

§ 371 Date: Mar. 3, 2009

(30) Foreign Application Priority Data
Sep. 7, 2006 (EP) 06120281.8

(51) Int. Cl. A61B 5/05

(52) U.S. Cl. 600/425

(57) ABSTRACT

The invention relates to a method (1) for imaging an interior of a turbid medium comprising the following steps: a) accommodation (3) of the turbid medium in a receiving volume comprising an entrance position for light and an exit position for light; b) irradiation (5) of the turbid medium with light from a light source using an entrance position for light; c) detection (10) of light emanating from the receiving volume as a result of the irradiation of the turbid medium using a photodetector unit using an exit position for light; d) detection (15, 17) of an out of bounds displacement of the turbid medium using light emanating from the receiving volume. To this end the invention proposes a number of ways to detect an out of bounds displacement of the turbid medium based on comparing at least two signals relating to light emanating from the receiving volume and a single exit position. The invention also relates to a device (45) for imaging an interior of a turbid medium (90) and a medical image acquisition device (105) both employing the method. According to the invention the device (45) for imaging an interior of a turbid medium (90) and the medical image acquisition device are adapted such that they further comprise displacement detection means according to the method and its embodiments according to the invention.

### FIELD OF INVENTION

The invention relates to a method for imaging an interior of a turbid medium comprising the following steps:

a) accommodation of the turbid medium in a receiving volume comprising at least one entrance position for light and at least one exit position for light;
b) irradiation of the turbid medium with light from a light source using the at least one entrance position for light;
c) detection of light emanating from the receiving volume as a result of the irradiation of the turbid medium using a photodetector unit using the at least one exit position for light.

The invention also relates to a device for imaging an interior of a turbid medium comprising:

a) a receiving volume for receiving the turbid medium comprising at least one entrance position for light and at least one exit position for light;
b) a light source for irradiating the turbid medium using the at least one entrance position for light;
c) a photodetector unit for detecting light emanating from the receiving volume as a result of the irradiation of the turbid medium using the at least one exit position for light.

The invention also relates to a medical image acquisition device comprising:

a) a receiving volume for receiving the turbid medium comprising at least one entrance position for light and at least one exit position for light;
b) a light source for irradiating the turbid medium using the at least one entrance position for light;
c) a photodetector unit for detecting light emanating from the receiving volume as a result of the irradiation of the turbid medium using the at least one exit position for light.

### BACKGROUND OF THE INVENTION

An embodiment of a method and device of this kind is known from U.S. Pat. No. 6,327,488 B1. The known method and device can be used for imaging an interior of a turbid medium such as biological tissue. In medical diagnostics the method and device may be used for imaging an interior of a female breast. The receiving volume receives a turbid medium, such as a breast. The turbid medium is then scanned by irradiating the turbid medium with light from a light source using an entrance position for light successively chosen from a plurality of entrance positions for light. Typically, light having a wavelength within the range of 400 nm to 1400 nm is used. As a result of irradiating the turbid medium light travels through the turbid medium in different directions. Light emanating from the receiving volume through a plurality of exit positions for light as a result of irradiating the turbid medium is detected using the photodetector unit resulting in a signal for each pair of entrance position and exit position for light per irradiation. The detected light is used to derive an image of an interior of the turbid medium.

### SUMMARY OF THE INVENTION

It is an object of the invention to further improve the quality of an image of an interior of the turbid medium.

According to the invention this object is realized in that the method further comprises the following additional step:

d) detection of an out of bounds displacement of the turbid medium using light emanating from the receiving volume.

The invention is based on the recognition that displacement of a turbid medium during a measurement leads to a loss of quality in a reconstructed image of an interior of the turbid medium. After all, if the turbid medium is displaced during a measurement a volume element of the turbid medium cannot be uniquely positioned during image reconstruction. Moreover, a change in the position of the turbid medium inside the receiving volume changes the lighting conditions inside the receiving volume and consequently also the light emanating from the receiving volume. By looking for an out of bounds displacement of the turbid medium during a measurement, a measurement can be stopped and a new measurement started if an out of bounds displacement is indeed detected. In this way, displacement of the turbid medium during a measurement can be kept within defined limits resulting in better control over image quality.

It is an additional advantage of the invention that detection of an out of bounds displacement of the turbid medium during a measurement may also be used as a safety measure. In medical diagnostics where the known method and device may be used for imaging an interior of, for instance, a female breast the light source may be arranged to emit laser light. If the patient moves during a measurement the risk of the patient being injured by the laser light, for instance by the patient looking into the laser light, cannot be completely ruled out. However, by detecting displacement of the turbid medium, in this case a patient's breast, during a measurement and to stop the measurement if the detected displacement is out of bounds, the laser light can be prevented from injuring the patient.

An embodiment of the method according to the invention comprises detection of a possible out of bounds displacement of the turbid medium that is based on comparing at least two signals relating to light emanating from the receiving volume at one of the exit positions for at least one of the exit positions. A displacement of the turbid medium inside the receiving volume will change the lighting conditions inside the receiving volume and consequently also the light emanating from the receiving volume. Hence, by comparing at least two signals relating to light emanating from the receiving volume at a single exit position, it can be determined whether the turbid medium has changed its position in the time period between which the at least two signals were obtained. This embodiment has the advantage that it is easy to implement, because it only depends on light emanating from the receiving volume in order to detect an out of bounds displacement of the turbid medium. Hence, necessary changes to the known method and device will be minimal.

In a further embodiment of the method according to the invention the at least two signals are obtained by dividing a single signal obtained by detecting light emanating from the receiving volume at a single one of the exit positions as a result of a single irradiation into at least two sets. Comparing the at least two signals enables detection of an out of bounds displacement of the turbid medium inside the receiving volume. This embodiment has the advantage that it is easy to implement. Furthermore, it does not require irradiation of the turbid medium using multiple entrance positions sequentially. Hence, there is no need for optical switching.

In a further embodiment of the method according to the invention the at least two signals are obtained by irradiating the turbid medium at least twice from a single one of the entrance positions. This embodiment has the advantage that it is easy to implement.

In a further embodiment of the method according to the invention the at least two signals are obtained by at least twice detecting light emanating from the receiving volume when the turbid medium is not irradiated. This embodiment has the advantage that it is easy to implement. The measurement process remains unchanged. In theory no light emanates from the receiving volume when the turbid medium is not irradiated. However, there may be an offset in the detection system. Hence, an offset measurement is done at the start of a scan. This offset measurement may be repeated during the scan. The offset measurement forms the first of the at least two signals. If a significant change is detected in another signal relating to the amount of light emanating from the receiving volume it may be assumed that the turbid medium has experienced an out of bounds displacement and that environmental light is able to reach the detector system.

In a further embodiment of the method according to the invention the at least two signals relate to the detection of a displacement flagging signal and a reference signal relating to the displacement flagging signal. A displacement flagging signal may be coupled into the receiving volume with the displacement flagging signal being chosen such that it is not detected at least one exit position after the initial accommodation of the turbid medium inside the receiving volume. However, if the turbid medium experiences an out of bounds displacement the lighting conditions inside the receiving volume are changed such that the flagging signal is detected at an exit position at which the flagging signal was not detected prior to the out of bounds displacement. This embodiment has the advantage that it is easy to implement as a possible flagging signal may be light having a wavelength such that the light is not transmitted through the turbid medium.

It is an object of the invention to provide a device for imaging an interior of a turbid medium according to the opening paragraphs that further improves the quality of an image of an interior of the turbid medium. This object is realizing that the device further comprises:

d) displacement detection means for detecting an out of bounds displacement of the turbid medium using light emanating from the receiving volume according to the method or any of the embodiments of the method according to the invention.

If, for instance, the device is used for imaging an interior of a female breast, as is done in medical diagnostics, the device benefits from any of the previous embodiments.

It is an object of the invention to provide a medical image acquisition device according to the opening paragraphs that further improves the quality of an image of an interior of a turbid medium. This object is realizing that the medical image acquisition device further comprises:

d) displacement detection means for detecting an out of bounds displacement of the turbid medium using light emanating from the receiving volume according to the method or any of the embodiments of the method according to the invention.

If, for instance, the medical image acquisition device is used for imaging an interior of a female breast, as is done in medical diagnostics, the medical image acquisition device benefits from any of the previous embodiments.

### BRIEF DESCRIPTION OF THE DRAWINGS

These and other aspects of the invention will be further elucidated and described with reference to the drawings, in which:

FIG. 1 schematically shows a number of embodiments of the method according to the invention;

FIG. 2 schematically shows an embodiment of a device for imaging an interior of a turbid medium according to the invention;

FIG. 3 schematically shows an embodiment of a medical image acquisition device according to the invention.

### DETAILED DESCRIPTION OF EMBODIMENTS

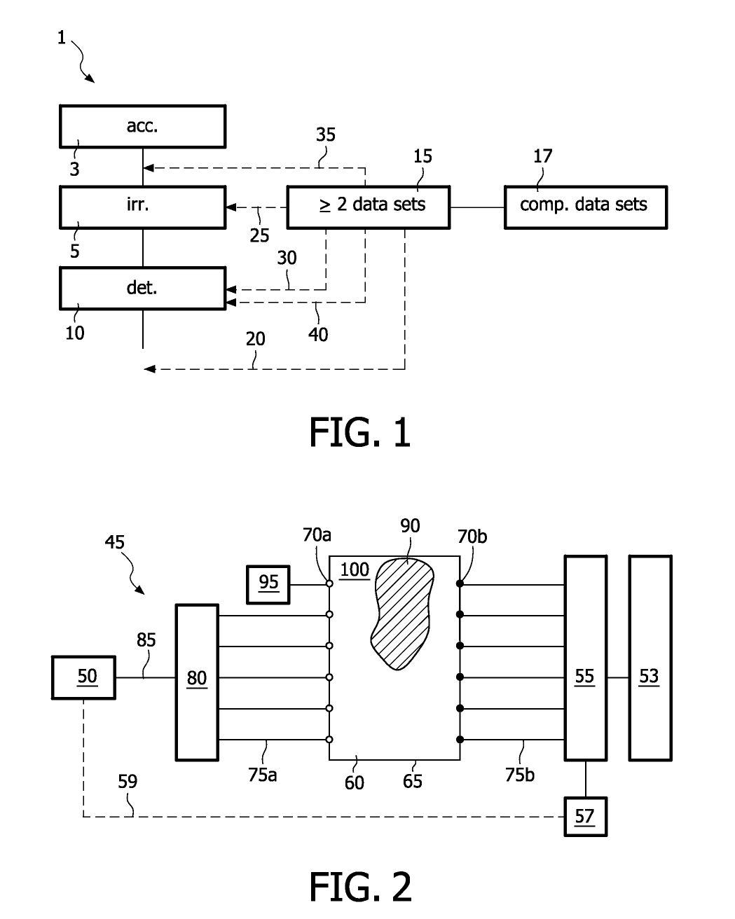

FIG. 1 schematically shows a number of embodiments of the method 1 according to the invention. In step 3 a turbid medium is accommodated in a receiving volume. In step 5 the turbid medium is irradiated with light from a light source by coupling light from the light source into the receiving volume using an entrance position for light. In step 10 light emanating from the receiving volume through an exit position for light as a result of irradiating the turbid medium with light from the light source is detected using a photodetector unit. As a result of step 15 at least two signals relating to light emanating from the receiving volume through the exit position are obtained which are then compared in order to detect an out of bounds displacement of the turbid medium. Several embodiments of step 15 are possible.

Dashed arrow 20 illustrates one such embodiment. In this embodiment the at least two signals are obtained by dividing a single signal obtained by detecting light emanating from the receiving volume at a single exit position during a single measurement into at least two sets. This embodiment has the advantage that the measurement process remains essentially unchanged. The measurement process follows the known method and only after data has been obtained this data is divided into at least two sets.

Dashed arrow 25 illustrates a further embodiment of step 15. In this embodiment the at least two signals are obtained by irradiating the turbid medium at least twice from a single entrance position. Irradiating the turbid medium at least twice from a single entrance position results in at least two signals being obtained at the photodetector unit, one set for each irradiation, which are then compared. The at least two irradiations of the turbid medium from a first entrance position can take place with or without the turbid medium being irradiated from another entrance position in the period of time between the at least two irradiations from the first entrance position.

If the method according to the invention is used for improving the quality of a reconstructed image of an interior of a turbid medium, the option of irradiating the turbid medium from another entrance position in the time between two irradiations from another entrance position may be preferable. The light source may be switched through all available positions first, after which only the first position is repeated to check for a displacement of the turbid medium.

However, if the at least two irradiations of the turbid medium from an entrance position take place without any further irradiations of the turbid medium from another entrance position being carried out in the meantime, there is no need for switching the light source from that entrance position to another. If the method according to the invention is used as a safety measure, for instance to prevent a patient from being injured by laser light in medical applications, this option is preferable as it does not require switching the light source from one position to another on timescales small enough to detect motion for safety purposes. In medical applications, for instance, a patient may move on a timescale ranging from tens of milliseconds to 100 ms.

Dashed arrow 30 illustrates a further embodiment of step 15. In this embodiment the at least two signals are obtained by at least twice detecting light emanating from the receiving volume when the turbid medium is not irradiated with light from the light source. An offset measurement, i.e. a measurement of the output of the photodetector unit when the turbid medium is not irradiated, is carried out at the start of a scan. At this moment a skilled operator using the method according to the invention monitors a stable and safe situation. The offset measurement may be repeated regularly during the scan and generates the first of the at least two signals. A further signal is obtained by performing another measurement of light emanating from the receiving volume when the turbid medium is not irradiated with light from the light source. The at least two signals may then be compared in order to determine an out of bounds displacement of the turbid medium. Dashed arrows 35 and 40 illustrate a further embodiment of step 15. In this embodiment the at least two signals relate to the detection of a displacement flagging signal and a reference signal relating to the displacement flagging signal. A displacement flagging signal may be coupled into the receiving volume with the displacement flagging signal being chosen such that it is not detected at least one exit position after the initial accommodation of the turbid medium inside the receiving volume. In medical diagnostics the method may be used for imaging an interior of a female breast. In that case the breast may be positioned in a receiving volume bound by a cup-like receptacle. The volume left inside the receptacle after the breast has been accommodated in the receiving volume may be filled with an optical matching fluid having optical properties that are similar to the optical properties of the turbid medium inside the receiving volume, in this case a patient's breast. A possible displacement flagging signal may then come from a light source that emits light that is substantially reflected by the matching fluid and the turbid medium and that is coupled into the receiving volume near the brim of the cup-like receptacle. If the patient moves such that the turbid medium is at least partially retracted from the receiving volume, the level of the matching fluid inside the receptacle will drop. Then the displacement flagging signal will be picked up by a detector inside the receiving volume that is positioned near the brim of the receptacle as the displacement flagging signal is no longer reflected by the matching fluid. This embodiment requires an offset measurement during which the output of the photodetector unit is determined when the turbid medium is accommodated in the receiving volume, the rest of the receiving volume is filled with matching fluid, and the displacement flagging signal is coupled into the receiving volume. This offset measurement may then be compared to a further measurement of the amount of displacement flagging signal emanating from the receiving volume during a scan.

In step 17 the at least two signals obtained in step 15 are compared in order to look for an out of bounds displacement of the turbid medium. In medical diagnostics where the known method may be used for, for instance, imaging an interior of a female breast, light emanating from the receiving volume through an exit position for light is detected essentially by counting photons. The obtained signal is a time-integrated signal representing the accumulated number of photons detected during the period of time over which the integration is carried out. Comparing at least two signals then means determining the difference, absolute or relative, between the at least two signals. The absolute difference between two signals can be obtained by subtracting one signal from another signal. The relative difference between two signals can be obtained by dividing the absolute difference through one of the signals between which the relative difference is being determined.

If the difference, absolute or relative, between at least two signals exceeds a predetermined threshold the turbid medium has experienced an out of bounds displacement. The lower limit of the predetermined threshold will be limited by the available signal to noise ratio. The threshold-value for the difference between two signals must be large enough so that the chance that a determined difference is insignificant due to the level of noise is acceptable. Acceptable means that, taking into account the number differences that are determined during a scan of a turbid medium, the chance that a scan will be terminated because of an insignificant difference is negligible. In other words, as the number of signals between which the difference is determined increases, the difference characterized by the noise level must increase accordingly. The same holds for an increase in the noise level.

The noise level can be characterized by the root mean square value of the noise on a signal. If the root mean square value of the noise characterizing the noise on two signals is R, then the difference between the two signals, expressed in units of R, must be large enough for the difference to be significant and for the chance that a scanned is terminated because an insignificant difference is negligible. Hence, as the number of signals for which the difference is determined increases, or as the noise level, characterized by its root mean square value, increases, the threshold-value for the difference at which a scan is terminated, expressed in units of R, increases. The known device comprises 256 entrance positions for light and 256 exit positions for light. During a scan of a turbid medium the 256 entrance positions are used sequentially, whereas all 256 exit positions simultaneously are used for each entrance position. If during a scan the difference between at least two signals is determined for each of the exit positions, then the number determined differences exceeds 65,000. The lower limit for a significant threshold-value can be expected to be 10R.

If the method according to the invention is used not as a safety measure but as a method for improving the quality of a reconstructed image of an interior of a turbid medium, the continuously updated average value of the differences determined during a scan, or a representation thereof, can be displayed on a screen comprised in the device. Based on the displayed value, an operator of the device can then decide to terminate a scan if he knows from experience that a value above a certain threshold will not result in optimal image quality.

FIG. 2 schematically shows an embodiment of a device 45 for imaging an interior of a turbid medium according to the invention. The device 45 comprises a light source 50, a photodetector unit 55, an image reconstruction unit 53 for reconstructing an image of an interior of a turbid medium 90 based on light detected using the photodetector unit 55. The device 45 further comprises a measurement volume 60 bound by a receptacle 65, said receptacle comprising a plurality of entrance positions for light 70a and exit positions for light 70b, and light guides 75a and 75b coupled to said entrance positions and exit positions for light. The device 45 further comprises a selection unit 80 for coupling the input light guide 85 to a number of entrance positions selected from the plurality of entrance positions 70a in the receptacle 65. For the sake of clarity, entrance positions 70a and exit positions 70b have been positioned at opposite sides of the receptacle 65. In reality, however, both entrance positions and exit positions may be distributed around the measurement volume 60. The turbid medium 90 is placed inside the measurement volume 60. The turbid medium 90 is then irradiated with light from the light source 50 from a plurality of positions by coupling the light source 50 using the selection unit 80 to successively selected entrance positions 70a. The light is chosen such that it is capable of propagating through the turbid medium 90. If, as may be the case in medical diagnostics, the device 45 is used for imaging an interior of a female breast, suitable light is, for instance, laser light with a wavelength within the range of 650 nm to 900 nm. Light emanating from the measurement volume 60 as a result of irradiating the turbid medium 90 is detected from a plurality of exit positions using exit positions 70b and using photodetector 55. The detected light is then used to derive an image of an interior of the turbid medium 90. Deriving an image of an interior of the turbid medium 90 based on the detected light is possible as at least part of this light has traveled through the turbid medium 90 and, as a consequence, contains information relating to an interior of the turbid medium 90. FIG. 2 illustrates one embodiment of the method according to the invention as illustrated in FIG. 1. In FIG. 2 the device 45 comprises a light source 95 emitting a displacement flagging signal that is coupled into the receiving volume 60. At least a part of the volume inside the receiving volume 60 that is not occupied by the turbid medium 90 is filled with the optical matching fluid 100 that has optical properties that are similar to those of the turbid medium 90. The light emitted by the light source 95 is chosen such that it is reflected by the matching fluid 100 and the turbid medium 90. Hence, without a displacement of the turbid medium 90 the light emitted by the light source 95 will basically not be detected except for a possible offset. To determine this offset an offset measurement is carried out. If the turbid medium 90 is displaced such that it is at least partially retracted from the receiving volume 60 the level of the matching fluid 100 inside the receiving volume 60 will drop and the displacement flagging signal will be detected by the photodetector unit 55. The device 45 further comprises a computer unit 57 which is used to compare the measured level of the displacement flagging signal to the level of the offset measurement. In general, computer unit 57 is used to determine whether the turbid medium 90 has experienced an out of bounds displacement by comparing the at least two signals relating to light emanating from the receiving volume 60 obtained according to any of the embodiments of the invention as discussed in relation to FIG. 1. Computer unit 57 may also be used for controlling the light source 50, as indicated by the dashed arrow 59, for instance if the light source 50 is used for irradiating the turbid medium 90 at least twice from a single one of the entrance positions 70a. In FIG. 2 the measurement volume 60 is bound by a receptacle 65. However, this need not always be the case. Another embodiment of a device for imaging an interior of a turbid medium is that of a handheld device that may, for instance, be pressed against a side of a turbid medium. In that case, the measurement volume is the volume occupied by the part of the turbid medium from which light is detected as a result of irradiating the turbid medium.

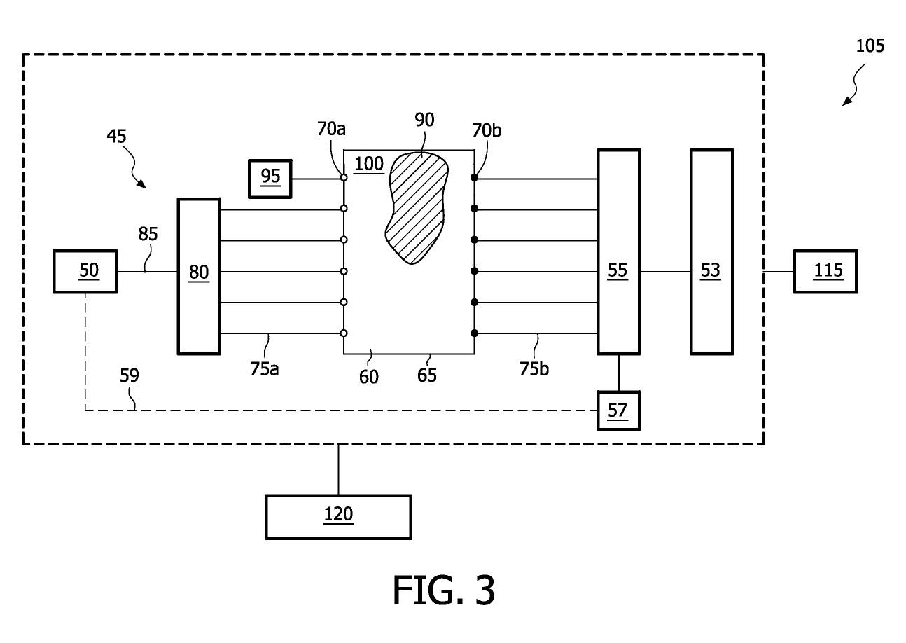

FIG. 3 schematically shows an embodiment of a medical image acquisition device according to the invention. The medical image acquisition device 105 comprises the device 45 discussed in FIG. 2 indicated by the dashed square. The medical image acquisition device 105 further comprises a screen 115 for displaying an image of an interior of the turbid medium 90 and an input interface 120, for instance, a keyboard enabling an operator to interact with the medical image acquisition device 105.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. The word “comprising” does not exclude the presence of elements or steps other than those listed in a claim. The word “a” or “an” preceding an element does not exclude the presence of a plurality of such elements. In the system claims enumerating several means, several of these means can be embodied by one and the same item of computer readable software or hardware. The mere fact that certain measures are recited in mutually different dependent claims does not indicate that a combination of these measures cannot be used to advantage.

1. A method (1) for imaging an interior of a turbid medium (90) comprising the following steps:
a) accommodation (3) of the turbid medium (90) in a receiving volume (60) comprising at least one entrance position (70a) for light and at least one exit position (70b) for light;
b) irradiation (5) of the turbid medium (90) with light from a light source (50) using the at least one entrance position (70a) for light;
c) detection (10) of light emanating from the receiving volume (60) as a result of the irradiation (5) of the turbid medium (90) using a photodetector unit (55) using the at least one exit position (70b) for light;
d) detection (15, 17) of an out of bounds displacement of the turbid medium (90) using light emanating from the receiving volume (60).

2. A method (1) as claimed in claim 1, wherein detection of an out of bounds displacement of the turbid medium (90) is based on comparing at least two signals relating to light emanating from the receiving volume (60) at one of the exit positions (70b) for at least one of the exit positions (70b).

3. A method (1) as claimed in claim 2, wherein the at least two signals are obtained by dividing a single signal obtained by detecting light emanating from the receiving volume (60) at a single one of the exit positions (70b) as a result of a single irradiation into at least two sets.

4. A method (1) as claimed in claim 2, wherein the at least two signals are obtained by irradiating the turbid medium (90) at least twice from a single one of the entrance positions (70a).

5. A method (1) as claimed in claim 2, wherein the at least two signals are obtained by at least twice detecting light emanating from the receiving volume (60) when the turbid medium (90) is not irradiated.

6. A method (1) as claimed in claim 2, wherein the at least two signals relate to the detection of a displacement flagging signal and a reference signal relating to the displacement flagging signal.

7. A device (45) for imaging an interior of a turbid medium (90) comprising:
a) a receiving volume (60) for receiving the turbid medium (90) comprising at least one entrance position (70a) for light and at least one exit position (70b) for light;
b) a light source (50) for irradiating the turbid medium (90) using the at least one entrance position (70a) for light;
c) a photodetector unit (55) for detecting light emanating from the receiving volume (60) as a result of the irradiation of the turbid medium (90) using the at least one exit position (70b) for light;
d) displacement detection means (55, 57) for detecting an out of bounds displacement of the turbid medium (90) using light emanating from the receiving volume (60).

8. A medical image acquisition device (105) comprising:
a) a receiving volume (60) for receiving the turbid medium (90) comprising at least one entrance position (70a) for light and at least one exit position (70b) for light;
b) a light source (50) for irradiating the turbid medium (90) using the at least one entrance position (70a) for light;
c) a photodetector unit (55) for detecting light emanating from the receiving volume (60) as a result of the irradiation of the turbid medium (90) using the at least one exit position (70b) for light;
d) displacement detection means (55, 57) for detecting an out of bounds displacement of the turbid medium (90) using light emanating from the receiving volume (60).
